(19) United States
(12) Patent Application Publication
(10) Pub. No.: US 2025/0218166 A1
(43) Pub. Date: Jul. 3, 2025

(54) Measuring Water Concentration in a Liquid Medium

(71) Applicant: Saudi Arabian Oil Company, Dhahran (SA)

(72) Inventors: Maha Nour, Thuwal (SA); Khawlah Kharashi, Thuwal (SA); Nada Ibrahim Alruwaii, Thuwal (SA); Ali Saad Aldossary, Al Khubar (SA); Diki Andrian, Ras Tanura (SA); Ali Alshehri, Thuwal (SA)

(21) Appl. No.: 18/402,186

(22) Filed: Jan. 2, 2024

(51) Int. Cl. G06V 10/82; G01N 21/17; G01N 21/3577

(52) U.S. Cl. CPC G06V 10/82; G01N 21/3577; G01N 2021/177

(57) ABSTRACT

Systems and methods for measuring water concentration in a liquid medium include a flow cell configured to fluidly couple to a pipe; a light source coupled to a first side of the flow cell; an infrared camera coupled to a second side of the flow cell opposite the first side; and at least one processor and a memory storing instructions that when executed by the at least one processor cause the at least one processor to perform operations. The operations include acquiring one or more images from the infrared camera; detecting water droplets in the one or more images using a trained machine learning model; and determining a concentration of water in the liquid medium based on the detected water droplets.

### TECHNICAL FIELD

This disclosure generally relates to measuring water content in a liquid medium.

### BACKGROUND

In the oil and gas industry, water is often produced from hydrocarbon wells along with the target hydrocarbons (e.g., crude oil, natural gas) being extracted. During downstream processing of the extracted hydrocarbons, the water is separated from the hydrocarbons. Measuring the water concentration in liquid hydrocarbon streams (e.g., hydrocarbon condensates, natural gas liquids) is useful to determine a quality of the hydrocarbon product and gives an indication of the efficacy of the separation of the water from the hydrocarbons and whether additional separation is necessary.

### SUMMARY

Free water (e.g., undissolved water) in oil separates naturally due to buoyancy in ambient conditions such as standard atmospheric pressure and temperature. When oil and free water have an elevated pressure, elevated temperature, high flow rate, or high degree of mixing, the free water may not separate naturally. The water can be mixed with the hydrocarbons in the form of small droplets making it challenging to quantify the volume of water in the liquid hydrocarbon medium.

This disclosure describes systems and methods for measuring water concentration in a liquid medium. A system includes a flow cell configured to be coupled to a pipe carrying the liquid medium (e.g., hydrocarbon condensate, natural gas liquids). An infrared camera is coupled to one side of the flow cell. A light source is coupled to a side of the flow cell opposite the infrared camera. A data processing system (e.g., a computer or control system) acquires one or more images from the infrared camera. The data processing system detects water droplets in the images using a trained machine learning model. The data processing system determines a concentration of water in the liquid medium based on the detected water droplets.

Implementations of the systems and methods of this disclosure can provide various technical benefits. The data processing system can differentiate between water droplets and other droplets, bubbles, or particles based on the infrared emissivity difference between the various substances. The water concentration can be determined in place without a need to take samples from a pipeline. Low concentrations of water (e.g., 20 ppm) can be detected. The water concentration of the liquid medium can be monitored on a continuous basis by acquiring and processing time-series of images from the infrared camera. Detection of water concentration above a threshold value can lead to early mitigation of environmental conditions that could lead to corrosion or damage of the pipeline or other equipment. In addition to measuring water concentrations in hydrocarbon condensate, the systems and methods of this disclosure can be adapted for other applications, for example, water in oil, oil in water, or for monitoring concentrations of metals in a liquid medium.

The details of one or more implementations of these systems and methods are set forth in the accompanying drawings and the description below. Other features, objects, and advantages of these systems and methods will be apparent from the description and drawings, and from the claims.

### DESCRIPTION OF DRAWINGS

FIG. 1 is a schematic diagram of a system for measuring water concentration in a liquid medium.

FIG. 2 is a schematic diagram of another system for measuring water concentration in a liquid medium.

FIG. 3 is a flow chart of a method for measuring water concentration in a liquid medium using the systems of FIG. 1 or 2.

FIG. 4 is a block diagram illustrating an example computer system used to provide computational functionalities associated with described algorithms, methods, functions, processes, flows, and procedures according to some implementations of the present disclosure.

Like reference symbols in the various drawings indicate like elements.

### DETAILED DESCRIPTION

This specification describes systems and methods for measuring water concentration in a liquid medium. A system includes a flow cell configured to be coupled to a pipe carrying the liquid medium (e.g., hydrocarbon condensate, natural gas liquids). An infrared camera is coupled to one side of the flow cell. A light source is coupled to a side of the flow cell opposite the infrared camera. A data processing system (e.g., a computer or control system) acquires one or more images from the infrared camera. The data processing system detects water droplets in the images using a trained machine learning model. The data processing system determines a concentration of water in the liquid medium based on the detected water droplets.

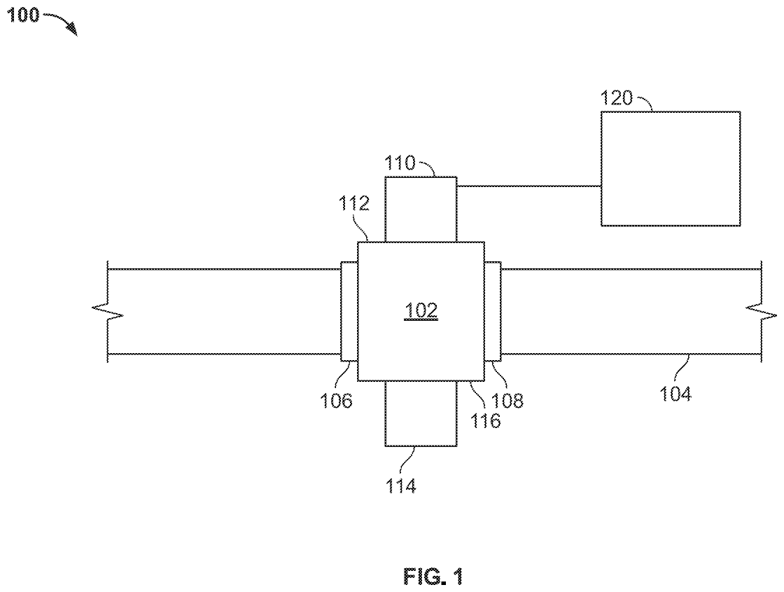

FIG. 1 is a schematic diagram of an example system 100 for measuring water concentration in a liquid medium. System 100 includes a flow cell 102. The flow cell 102 forms the measurement region for the system 100. The flow cell 102, as depicted in FIG. 1, is coupled in line with a pipe 104. The flow cell 102 is coupled to the pipe by flanges 106, 108. An infrared camera 110 is coupled to a first side 112 of the flow cell 102. A light source 114 (e.g., an infrared light source) is coupled to a second side 116 of the flow cell. The first side 112 and the second side 116 are opposite one another on the flow cell 102. The first side 112 and the second side 116 are transparent or include transparent portion to enable the infrared camera 110 and the light source 114 to transmit and receive light through the flow cell 102. In some implementations, magnifying lenses are coupled to the transparent portions to magnify the flow of the liquid medium through the flow cell 102.

Liquid flowing through pipe 104 also flows through the flow cell 102. The liquid can be, for example, hydrocarbon condensates, C3+ natural gas liquids (NGL), or both. C3+ NGL includes natural gas liquids with molecules having three or more carbon atoms, e.g., propane, butane, pentane, hexane, heptane, and so forth. The carbon atoms can be single, double, or triple bonded or other arrangement (e.g., alkanes, alkenes, alkynes, cycloalkanes, alkadienes). The liquid medium flowing through the pipe 104 can have elevated pressure (e.g., 400 psi or more) and temperature (e.g., 180 degrees Fahrenheit (° F.) or more) relative to typical atmospheric conditions. The elevated temperature and pressures in the pipe make it impractical to take samples from the pipe to measure the water concentration as the water can evaporate at the higher temperature at atmospheric pressure. The liquid can also have high flow rate (e.g., 60 million barrels per day (MBPD)) The high flow rate of the liquid can contribute to mixing of the liquid medium with the water and breakup of water droplets into smaller water droplets.

The system 100 also includes a data processing system 120 in communication with the infrared camera 110. The data processing system 120 includes at least one processor and memory storing instructions for measuring the water concentration in the liquid medium. The data processing system 120 can be for example a controller or computer. The data processing system can communicate with the camera or other components of the system 100 via wired (e.g., ethernet, serial communication link, USB) or wireless connections (e.g., Wi-Fi, Bluetooth, cellular).

The system 100 is capable of measuring low water concentrations (e.g., 100 parts per million (ppm) or less, 20 ppm or less, 1 ppm or more). The size of water droplets that can be detected by the system 100 can depend on the optics (e.g., lenses) that the infrared camera 110 is equipped with. For example, when the infrared camera 110 is equipped with a microscope objective, the system 100 can detect micro-scale water droplets. The optics for the infrared camera can be adapted based on the needs of particular applications.

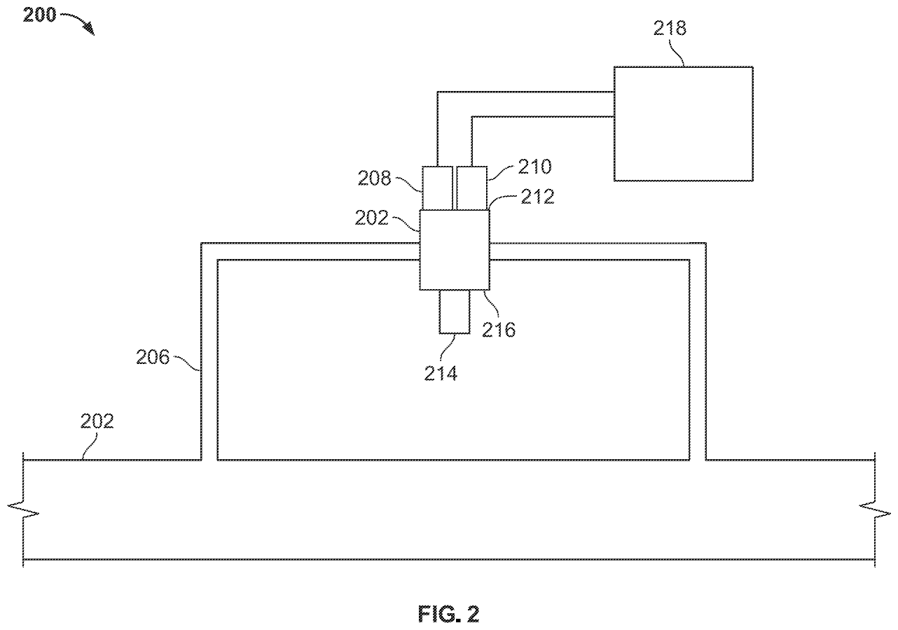

FIG. 2 is a schematic diagram of another example system 200 for measuring water concentration in a liquid medium. System 200 includes a flow cell 202 configured to receive the liquid medium from pipe 204 via inflow loop 206. An inflow loop 206 can be used, for example, in systems having a pipe with a large diameter (e.g., 1 inch or more, 5 inches or more, 10 inches or more). The inflow loop 206 has a smaller diameter than the pipe 204. An infrared camera 208 and a visible light camera 210 are coupled to a first side 212 of the flow cell 202. A light source 214 is coupled to a second side 216 of the flow cell 202. The second side 216 is opposite the first side 212. The light source 214 is operable to emit light in the visible spectrum and in the infrared spectrum.

The infrared camera 208 and the visible light camera 210 are communicatively coupled to a data processing system 218. The data processing system 218 can acquire images from both the infrared camera 208 and the visible light camera 210. Images taken from the infrared camera 208 and the visible light camera 210 at the same instant in time and with the same or overlapping fields of view can be processed together to improve the detection of water droplets.

In some implementations, a system for measuring water concentration in a liquid medium can include features of system 100, system 200, or both. For example, a system coupled to a pipeline (as shown in FIG. 1) can include both a visible light camera and an infrared camera. Likewise, a system coupled to an inflow loop (as shown in FIG. 2) can include an infrared camera without including a visible light camera.

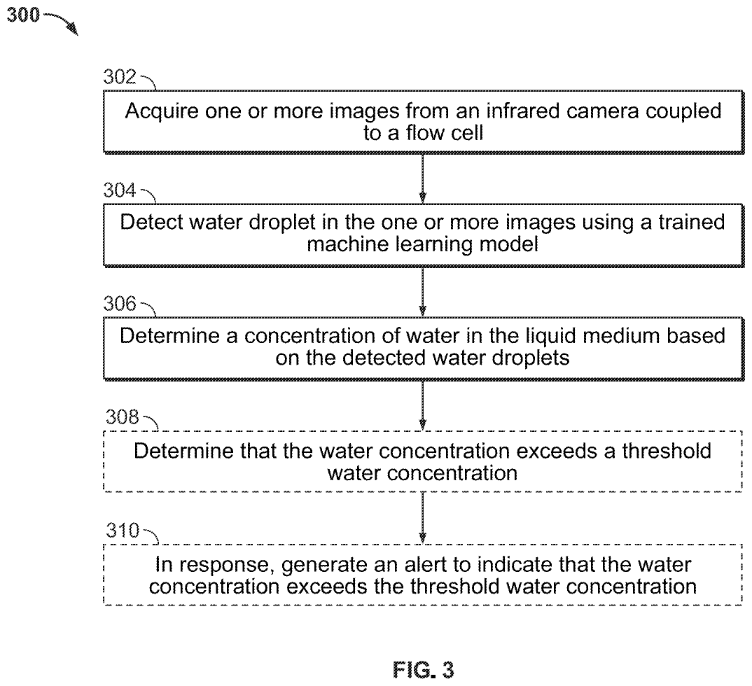

FIG. 3 is a flow chart for an example method 300 for measuring water concentration in a liquid medium. The method 300 can be implemented on a data processing system such as a computer or control system (e.g., data processing system 120 or data processing system 218). The data processing system can be located near to the measurement location or remote from the measurement location.

The data processing system acquires one or more images from an infrared camera coupled to a flow cell including a liquid medium (step 302). The liquid medium can include hydrocarbon condensates, C3+ natural gas liquids, or both. The one or more images can be acquired as a time series of images (e.g., a video). For example, images of a time-series of images can be separated in time by a fixed time interval. Analyzing a time-series of images can produce, for example, information about the time rate of change of water concentration in the liquid medium.

The data processing system detects water droplets in the one or more images using a trained machine learning model (step 304). For example, the trained machine learning model can be a trained recurrent neural network. The trained machine learning model can take as input an image from the infrared camera. The trained machine learning model generates as output locations, sizes, or both, of detected water droplets in the input image. The trained machine learning model can remove unwanted data (e.g., gas bubbles or solid particles) from the images to isolate water droplets in the images.

In some implementations, the data processing system preprocesses the one or more images acquired from the infrared camera to remove the unwanted data before detecting the water droplets using the trained machine learning model. For example, the data processing system can use computer vision techniques like object detection and image filtering to remove unwanted bubbles, particles, and noise from the images.

In some implementations, the machine learning model can take as input process conditions. For example, inputs to the machine learning model can include physical properties of the liquid medium (e.g., viscosity, density, surface tension, etc.), temperature, pressure, and flow rate.

The data processing system can train the machine learning model using a training data set. The training dataset can include images acquired from the infrared camera having water droplets in the images labeled. In some implementations, the data processing system generates synthetic data for the training data set. For example, the data processing system can perform a fluid dynamic simulation of a liquid medium including water droplets. The data processing system can generate synthetic images based on the fluid dynamic simulation.

Based on the detected water droplets, the data processing system determines a concentration of water in the liquid medium (step 306). For example, the detected water droplets can appear as circles in the two dimensional images. The data processing system can determine a volume of water associated with each detected droplet based on a measured diameter of the two dimensional circle representing the droplet. The volumes of each detected water droplet can be combined to determine an overall volume of water detected in the image. The data processing system can determine the concentration of water based on the overall volume of water and a volume associated with the field of view of the image.

In some implementations, the data processing system determines that the concentration of water exceeds a threshold water concentration (step 308). The threshold water concentration can be, for example, a user defined value or based on the materials of the pipe or processing facility.

In response to determining that the concentration of water exceeds a threshold water concentration, the data processing system can generate an alert to indicate that the water concentration exceeds the threshold water concentration (step 310). For example, the data processing system can generate an audible alert (e.g., siren or alarm), a visual alert (e.g., flashing light, pop-up message on a display device, etc.), or both. In some implementations, the data processing system can transmit a message to other computers or processing devices to cause the other devices to generate the alert.

In some implementations, in response to determining that the concentration of water exceeds the threshold water concentration, the data processing system generates commands to control fluid separation equipment to separate water from the liquid medium. For example, the data processing system can generate commands to control valves to direct the liquid medium to flow to equipment for further fluid separation. The measurement of water concentration can serve as a quality control factor, and the data processing system can divert the liquid medium having excess water (e.g., the concentration of water exceeds the threshold water concentration) to processing equipment to process the liquid medium.

In implementations including a visible light camera, the data processing system can acquire one or more images from the visible light camera. The data processing system can synchronize the acquisition of the images from the visible light camera and the infrared camera to enable the images to show the same or substantially the same instant in time. The data processing system can detect water droplets in the images from the visible light camera and the infrared camera using a machine learning model. In some implementations, a single machine learning model can take as input infrared and visible light images. In some implementations, multiple machine learning models can be used to process the images separately.

In some implementations, the data processing system continuously acquires images from the infrared camera and/or the visible light camera. For example, the data processing system acquires an image at a fixed rate (e.g., 1 or more image every 10 seconds, 1 or more image per second, 30 or more images per second).

In some implementations, the data processing system processes the images in real time. Real-time or near real-time processing and/or communication refers to a scenario in which received data (e.g., images) are processed as made available to systems and devices requesting those data immediately (e.g., within milliseconds, tens of milliseconds, or hundreds of milliseconds) after the processing of those data are completed, without introducing data persistence or store-then-forward actions. In this context, a real-time data processing system is configured to process an infrared image or a visible light image as it arrives and determine the concentration of water in the liquid as quickly as possible (though processing latency may occur). Though data can be buffered between module interfaces in a pipelined architecture, each individual module operates on the most recent data available to it. The overall result is a workflow that, in a real-time context, receives a data stream (e.g., images) and outputs processed data (e.g., water concentration) based on that data stream in a first-in, first out manner. However, non-real-time contexts are also possible, in which data are stored (either in memory or persistently) for processing at a later time. In this context, modules of the data processing system do not necessarily operate on the most recent data available.

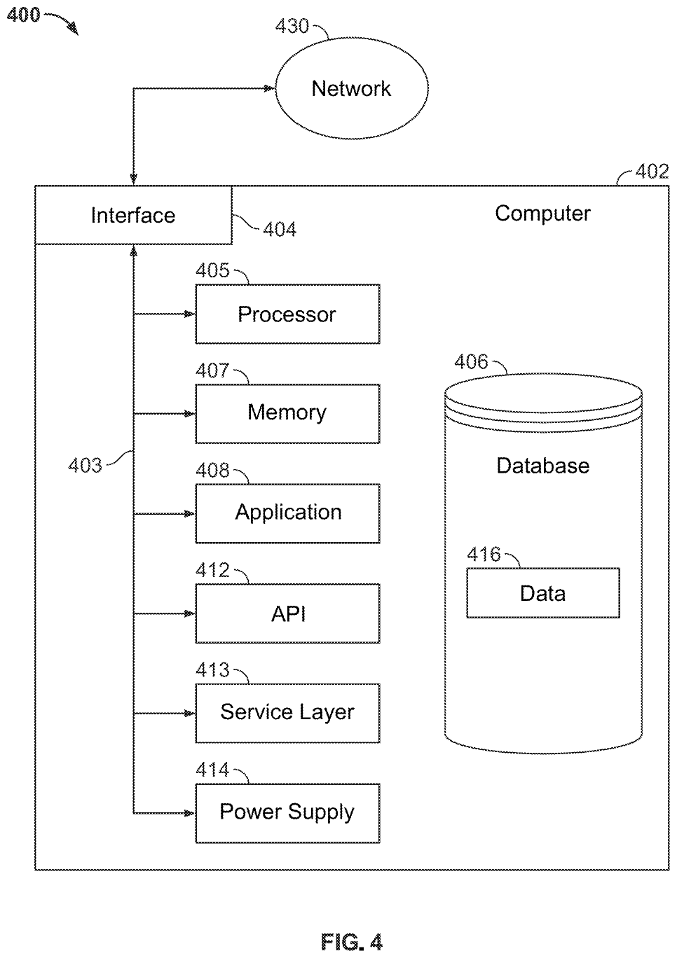

FIG. 4 is a block diagram of an example computer system 400 used to provide computational functionalities associated with described algorithms, methods, functions, processes, flows, and procedures described in the present disclosure, according to some implementations of the present disclosure. The illustrated computer 402 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 402 can include input devices such as keypads, keyboards, and touch screens that can accept user information. Also, the computer 402 can include output devices that can convey information associated with the operation of the computer 402. The information can include digital data, visual data, audio information, or a combination of information. The information can be presented in a graphical user interface (UI) (or GUI).

The computer 402 can serve in a role as a client, a network component, a server, a database, a persistency, or components of a computer system for performing the subject matter described in the present disclosure. The illustrated computer 402 is communicably coupled with a network 430. In some implementations, one or more components of the computer 402 can be configured to operate within different environments, including cloud-computing-based environments, local environments, global environments, and combinations of environments.

At a high level, the computer 402 is an electronic computing device operable to receive, transmit, process, store, and manage data and information associated with the described subject matter. According to some implementations, the computer 402 can also include, or be communicably coupled with, an application server, an email server, a web server, a caching server, a streaming data server, or a combination of servers.

The computer 402 can receive requests over network 430 from a client application (for example, executing on another computer 402). The computer 402 can respond to the received requests by processing the received requests using software applications. Requests can also be sent to the computer 402 from internal users (for example, from a command console), external (or third) parties, automated applications, entities, individuals, systems, and computers.

Each of the components of the computer 402 can communicate using a system bus 403. In some implementations, any, or all of the components of the computer 402, including hardware or software components, can interface with each other or the interface 404 (or a combination of both), over the system bus 403. Interfaces can use an application programming interface (API) 412, a service layer 413, or a combination of the API 412 and service layer 413. The API 412 can include specifications for routines, data structures, and object classes. The API 412 can be either computer-language independent or dependent. The API 412 can refer to a complete interface, a single function, or a set of APIs.

The service layer 413 can provide software services to the computer 402 and other components (whether illustrated or not) that are communicably coupled to the computer 402. The functionality of the computer 402 can be accessible for all service consumers using this service layer. Software services, such as those provided by the service layer 413, can provide reusable, defined functionalities through a defined interface. For example, the interface can be software written in JAVA, C++, or a language providing data in extensible markup language (XML) format. While illustrated as an integrated component of the computer 402, in alternative implementations, the API 412 or the service layer 413 can be stand-alone components in relation to other components of the computer 402 and other components communicably coupled to the computer 402. Moreover, any or all parts of the API 412 or the service layer 413 can be implemented as child or sub-modules of another software module, enterprise application, or hardware module without departing from the scope of the present disclosure.

The computer 402 includes an interface 404. Although illustrated as a single interface 404 in FIG. 4, two or more interfaces 404 can be used according to particular needs, desires, or particular implementations of the computer 402 and the described functionality. The interface 404 can be used by the computer 402 for communicating with other systems that are connected to the network 430 (whether illustrated or not) in a distributed environment. Generally, the interface 404 can include, or be implemented using, logic encoded in software or hardware (or a combination of software and hardware) operable to communicate with the network 430. More specifically, the interface 404 can include software supporting one or more communication protocols associated with communications. As such, the network 430 or the interface's hardware can be operable to communicate physical signals within and outside of the illustrated computer 402.

The computer 402 includes a processor 405. Although illustrated as a single processor 405 in FIG. 4, two or more processors 405 can be used according to particular needs, desires, or particular implementations of the computer 402 and the described functionality. Generally, the processor 405 can execute instructions and can manipulate data to perform the operations of the computer 402, including operations using algorithms, methods, functions, processes, flows, and procedures as described in the present disclosure.

The computer 402 also includes a database 406 that can hold data for the computer 402 and other components connected to the network 430 (whether illustrated or not). For example, database 406 can hold data 416. For example, database 406 can be an in-memory, conventional, or a database storing data consistent with the present disclosure. In some implementations, database 406 can be a combination of two or more different database types (for example, hybrid in-memory and conventional databases) according to particular needs, desires, or particular implementations of the computer 402 and the described functionality. Although illustrated as a single database 406 in FIG. 4, two or more databases (of the same, different, or combination of types) can be used according to particular needs, desires, or particular implementations of the computer 402 and the described functionality. While database 406 is illustrated as an internal component of the computer 402, in alternative implementations, database 406 can be external to the computer 402.

The computer 402 also includes a memory 407 that can hold data for the computer 402 or a combination of components connected to the network 430 (whether illustrated or not). Memory 407 can store any data consistent with the present disclosure. In some implementations, memory 407 can be a combination of two or more different types of memory (for example, a combination of semiconductor and magnetic storage) according to particular needs, desires, or particular implementations of the computer 402 and the described functionality. Although illustrated as a single memory 407 in FIG. 4, two or more memories 407 (of the same, different, or combination of types) can be used according to particular needs, desires, or particular implementations of the computer 402 and the described functionality. While memory 407 is illustrated as an internal component of the computer 402, in alternative implementations, memory 407 can be external to the computer 402.

The application 408 can be an algorithmic software engine providing functionality according to particular needs, desires, or particular implementations of the computer 402 and the described functionality. For example, application 408 can serve as one or more components, modules, or applications. Further, although illustrated as a single application 408, the application 408 can be implemented as multiple applications 408 on the computer 402. In addition, although illustrated as internal to the computer 402, in alternative implementations, the application 408 can be external to the computer 402.

The computer 402 can also include a power supply 414. The power supply 414 can include a rechargeable or non-rechargeable battery that can be configured to be either user- or non-user-replaceable. In some implementations, the power supply 414 can include power-conversion and management circuits, including recharging, standby, and power management functionalities. In some implementations, the power-supply 414 can include a power plug to allow the computer 402 to be plugged into a wall socket or a power source to, for example, power the computer 402 or recharge a rechargeable battery.

There can be any number of computers 402 associated with, or external to, a computer system containing computer 402, with each computer 402 communicating over network 430. Further, the terms “client,” “user,” and other appropriate terminology can be used interchangeably, as appropriate, without departing from the scope of the present disclosure. Moreover, the present disclosure contemplates that many users can use one computer 402 and one user can use multiple computers 402.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random access memory (RAM), read only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

A number of implementations of these systems and methods have been described. Nevertheless, it will be understood that various modifications may be made without departing from the spirit and scope of this disclosure. Accordingly, other implementations are within the scope of the following claims.

### Examples

In an example implementation, a system for measuring water concentration in a liquid medium includes a flow cell configured to fluidly couple to a pipe; a light source coupled to a first side of the flow cell; an infrared camera coupled to a second side of the flow cell opposite the first side; and at least one processor and a memory storing instructions that when executed by the at least one processor cause the at least one processor to perform operations including acquiring one or more images from the infrared camera; detecting water droplets in the one or more images using a trained machine learning model; and determining a concentration of water in the liquid medium based on the detected water droplets.

In an aspect combinable with the example implementation, the liquid medium includes hydrocarbon condensate, C3+ natural gas liquid, or both.

In another aspect combinable with any of the previous aspects, the trained machine learning model includes a trained recurrent neural network.

Another aspect combinable with any of the previous aspects includes a visible light camera coupled to the second side of the flow cell.

In another aspect combinable with any of the previous aspects, the operations include acquiring one or more images from the visible light camera; and detecting water droplets includes detecting water droplets in the one or more images from the infrared camera and the one or more images from the visible light camera using the trained machine learning model.

In another aspect combinable with any of the previous aspects, the flow cell includes flange connections to couple the flow cell to the pipe.

In another aspect combinable with any of the previous aspects, acquiring one or more images from the infrared camera includes acquiring a time-series of images where the images are separated in time by a fixed time interval.

In another example implementation, a method for measuring water concentration in a liquid medium includes acquiring one or more images from an infrared camera coupled to a flow cell including the liquid medium; detecting water droplets in the one or more images using a trained machine learning model; and determining a concentration of water in the liquid medium based on the detected water droplets.

In an aspect combinable with the example implementation, the liquid medium includes hydrocarbon condensate, C3+ natural gas liquid, or both.

In another aspect combinable with any of the previous aspects, the trained machine learning model includes a trained recurrent neural network.

Another aspect combinable with any of the previous aspects includes acquiring one or more images from a visible light camera coupled to the flow cell, and where detecting water droplets includes detecting water droplets in the one or more images from the infrared camera and the one or more images from the visible light camera using the trained machine learning model.

In another aspect combinable with any of the previous aspects, acquiring one or more images from the infrared camera includes acquiring a time-series of images where the images are separated in time by a fixed time interval.

Another aspect combinable with any of the previous aspects includes training the machine learning model based on images acquired from the infrared camera.

Another aspect combinable with any of the previous aspects includes generating synthetic training data to train the machine learning model, the synthetic training data includes images generated from a simulation of a flow of the liquid medium including water droplets.

Another aspect combinable with any of the previous aspects includes determining that the water concentration exceeds a threshold water concentration; and in response to determining that the water concentration exceeds the threshold water concentration, generating an alert to indicate that the water concentration exceeds the threshold water concentration.

Another aspect combinable with any of the previous aspects includes in response to determining that the water concentration exceeds the threshold water concentration, generating commands to control fluid separation equipment to separate water from the liquid medium.

In another example implementation, one or more non-transitory machine readable storage devices storing instructions for measuring water concentration in a liquid medium, the instructions being executable by one or more processors to cause performance of operations including acquiring one or more images from an infrared camera coupled to a flow cell including the liquid medium; detecting water droplets in the one or more images using a trained machine learning model; and determining a concentration of water in the liquid medium based on the detected water droplets.

In an aspect combinable with the example implementation, the operations include acquiring one or more images from a visible light camera coupled to the flow cell, where detecting water droplets includes detecting water droplets in the one or more images from the infrared camera and the one or more images from the visible light camera using the trained machine learning model.

In an aspect combinable with any of the previous aspects, acquiring one or more images from the infrared camera includes acquiring a time-series of images where the images are separated in time by a fixed time interval.

In an aspect combinable with any of the previous aspect, the operations include training the machine learning model based on images acquired from the infrared camera.

What is claimed is:

1. A system for measuring water concentration in a liquid medium, the system comprising:
a flow cell configured to fluidly couple to a pipe;
a light source coupled to a first side of the flow cell;
an infrared camera coupled to a second side of the flow cell opposite the first side; and
at least one processor and a memory storing instructions that when executed by the at least one processor cause the at least one processor to perform operations comprising:
acquiring one or more images from the infrared camera;
detecting water droplets in the one or more images using a trained machine learning model; and
determining a concentration of water in the liquid medium based on the detected water droplets.

2. The system of claim 1, wherein the liquid medium comprises hydrocarbon condensate, C3+ natural gas liquid, or both.

3. The system of claim 1, wherein the trained machine learning model comprises a trained recurrent neural network.

4. The system of claim 1, further comprising:
a visible light camera coupled to the second side of the flow cell.

5. The system of claim 4, wherein the operations further comprise:
acquiring one or more images from the visible light camera; and
wherein detecting water droplets comprises detecting water droplets in the one or more images from the infrared camera and the one or more images from the visible light camera using the trained machine learning model.

6. The system of claim 1, wherein the flow cell comprises flange connections to couple the flow cell to the pipe.

7. The system of claim 1, wherein acquiring one or more images from the infrared camera comprises acquiring a time-series of images wherein the images are separated in time by a fixed time interval.

8. A method for measuring water concentration in a liquid medium, the method comprising:
acquiring one or more images from an infrared camera coupled to a flow cell comprising the liquid medium;
detecting water droplets in the one or more images using a trained machine learning model; and
determining a concentration of water in the liquid medium based on the detected water droplets.

9. The method of claim 8, wherein the liquid medium comprises hydrocarbon condensate, C3+ natural gas liquid, or both.

10. The method of claim 8, wherein the trained machine learning model comprises a trained recurrent neural network.

11. The method of claim 8, further comprising:
acquiring one or more images from a visible light camera coupled to the flow cell,
wherein detecting water droplets comprises detecting water droplets in the one or more images from the infrared camera and the one or more images from the visible light camera using the trained machine learning model.

12. The method of claim 8, wherein acquiring one or more images from the infrared camera comprises acquiring a time-series of images, wherein the images are separated in time by a fixed time interval.

13. The method of claim 8, further comprising: training the machine learning model based on images acquired from the infrared camera.

14. The method of claim 13, further comprising: generating synthetic training data to train the machine learning model, the synthetic training data comprising images generated from a simulation of a flow of the liquid medium including water droplets.

15. The method of claim 8, further comprising: determining that the water concentration exceeds a threshold water concentration; and in response to determining that the water concentration exceeds the threshold water concentration, generating an alert to indicate that the water concentration exceeds the threshold water concentration.

16. The method of claim 15, further comprising: in response to determining that the water concentration exceeds the threshold water concentration, generating commands to control fluid separation equipment to separate water from the liquid medium.

17. One or more non-transitory machine readable storage devices storing instructions for measuring water concentration in a liquid medium, the instructions being executable by one or more processors to cause performance of operations comprising:
acquiring one or more images from an infrared camera coupled to a flow cell comprising the liquid medium;
detecting water droplets in the one or more images using a trained machine learning model; and
determining a concentration of water in the liquid medium based on the detected water droplets.

18. The one or more non-transitory machine readable storage devices of claim 17, the operations further comprise:
acquiring one or more images from a visible light camera coupled to the flow cell,
wherein detecting water droplets comprises detecting water droplets in the one or more images from the infrared camera and the one or more images from the visible light camera using the trained machine learning model.

19. The one or more non-transitory machine readable storage devices of claim 17, wherein acquiring one or more images from the infrared camera comprises acquiring a time-series of images wherein the images are separated in time by a fixed time interval.

20. The one or more non-transitory machine readable storage devices of claim 17, the operations further comprise:
training the machine learning model based on images acquired from the infrared camera.
